(19) United States
(12) Patent Application Publication
(10) Pub. No.: US 2021/0353851 A1
(43) Pub. Date: Nov. 18, 2021

(54) Treatment of Paranasal Sinus Disease

(71) Applicant: CYMBIDIUM MEDICAL LLC, Boulder, CO (US)

(72) Inventor: Harry Ross, Boulder, CO (US)

(73) Assignee: CYMBIDIUM MEDICAL LLC, Boulder, CO (US)

(21) Appl. No.: 17/284,095

(22) PCT Filed: Oct. 11, 2019
(86) PCT No.: PCT/US2019/055921

§ 371 Date: Apr. 9, 2021

Related U.S. Application Data
(60) Provisional application No. 62/744,786, filed on Oct. 12, 2018.

(51) Int. Cl. A61M 3/02; A61K 31/7036; A61K 31/573

(52) U.S. Cl. CPC A61M 3/0279; A61K 31/7036; A61M 2210/0681; A61M 2202/064; A61M 2210/0618; A61K 31/573

(57) ABSTRACT

A method for the treatment of paranasal sinus disease, administering a treatment formulation or a drug treatment formulation through the nasal cavity directly into a paranasal sinus.

### CROSS REFERENCE TO RELATED APPLICATIONS

This application claims benefit of U.S. provisional patent application No. 62/744,786 filed Oct. 12, 2018, the contents of which are incorporated herein by reference as if each and every portion thereof were set forth herein.

### FIELD OF THE INVENTION

The invention relates to treatment of symptoms and diseases of the paranasal sinus, including delivery of drugs, drug formulations and methods.

### BACKGROUND OF THE INVENTION

It is estimated that 35 million Americans are treated annually for sinus disease and symptoms associated with sinus disease. Sinusitis is a multifactorial disease characterized by inflammation of the mucous membranes lining the paranasal sinus cavities. The sinuses are air filled facial cavities which normally drain into the nasal cavity. In the disease state, the sinuses may become fluid filled if drainage is obstructed. Sinus disease can take many forms including acute, recurrent acute, chronic, infectious, allergic, polypoid and rhinitis. Some symptoms of sinus disease may include pain or tenderness around eyes, nose or forehead, headache, nasal discharge, sore throat, bad breath, nasal congestion, dental pain and fever. Treatment of sinus disease has an estimated cost of 12 to 18 billion dollars in the U.S. alone.

Current treatments for sinus disease and symptoms include both over the counter and prescription oral pills, nasal sprays and topical washes. For refractory disease various surgical options are available. For many patients, these therapies provide only limited or temporary relief, but rarely a cure.

Sinus disease represents just one form of disease of the respiratory tract, and more than 50 million Americans are treated annually for respiratory disease. The respiratory tract is broken into an upper respiratory system and the lower respiratory system. The upper respiratory system comprises the nasal cavity, paranasal sinuses, oral cavity, pharynx and larynx while the lower respiratory system comprises the trachea, bronchi which enter the lungs, bronchioles and alveoli). The upper and lower respiratory tract share a ciliated epithelium, one of whose purpose is to actively clear foreign particles including pathogens, allergens and debris. Often referred to as the muco-ciliary escalator, these tiny hair-like structures beat in unison, creating synchronized waves which propel particles off delicate structures for clearance to the throat and elimination through the GI tract. Nasal airways, paranasal sinuses, trachea, and pulmonary tree are all served by this mechanism. Without the mucociliary clearance mechanism foreign particles can lodge in the airway leading to infection, irritation, allergy and tissue injury. Diseases of the upper and lower respiratory tract creates an enormous annual medical and economic toll on patients and society. Fifteen percent of the population will seek medical treatment each year for sinus problems alone. Most patients will make an unremarkable recovery however 10-20% of those diagnosed with sinusitis will develop chronic rhinosinusitis (CRS) and enter a constant spiral of ill health, endless prescriptions, missed work and surgical procedures. Many will never be cured and require lifelong treatment.

Chronic rhinosinusitis is often considered to be an inflammatory disease. Treatment may include both oral and nasal spray steroids. Oral steroids have significant morbidity including increased risk of sepsis and osteoporosis/hip fracture. Nasal steroid sprays have fewer systemic side effects but have limited sinus penetration and often must be used daily for weeks or months at a time.

When infection presents, systemic antibiotics are generally prescribed. Often started for 10-14 days, it is not unusual for the prescription to be extended to 4-6 weeks. Existing oral medications are unable to adequately penetrate an intact sinus, usually delivering only microgram/ml tissue concentrations of active drug to the tissue. Oral dosing of any medication for sinus disease is problematic. GI absorption of oral medication is incomplete and a significant portion of any dose never makes it past the stomach barrier. Unique patient differences, timing to last meal, and other medications also drastically effect absorption. Upon transit through the stomach barrier, first pass effect occurring in the liver contributes to further loss. After passing the liver, the remaining drug is then broadly delivered to the full circulation and entire body. In the typical 70 kg individual, this is a massive dilution. Arbitrarily bathing all tissues, there is no specific concentration at the sinus or disease site over the rest of the body. In fact, the sinuses typically suffer from very poor drug diffusion. While most oral steroids, antibiotics, allergy medications etc. are dosed in hundreds of milligrams, measured drug concentration at the sinuses is usually only single digit microgram/cc concentration. While perhaps mildly effective, it is simply not possible to drive therapeutic concentration to the site of sinus disease. Reinfection often occurs and repetitive courses of antibiotics many times per year may be required. Of considerable concern is the resultant morbidity of systemic exposure to many of the commonly used drugs when oral dosing is used. Steroids can create severe depression, GI upset, glucose imbalance and considerable risk for both sepsis and osteoporosis/hip fracture. So common is infection that ˜20% of all antibiotics prescribed are for sinus disease alone. This frequent use leads to numerous side effects including stomach upset, diarrhea, and frequent yeast infections in women and puts individuals at risk for C. difficile infection. On a larger scale, the overuse of antibiotics is rapidly creating numerous drug resistant organisms for which doctors have limited viable treatments. Broad classes of medications are quickly becoming obsolete with no viable replacements in sight. On global scale, the stage is being set for a significant medical crisis as previously easily treated infectious diseases become resistant to existing medications.

Steroid and allergy nasal sprays are a mainstay of therapy for sino nasal disease but unfortunately, no antibiotic nasal spray exists. Relatively easy to use, moderately effective at delivery to the nasal mucosa, and with few systemic side effects they offer some advantages over oral dosing. On a broad level, many non sinus drugs are also delivered nasally for indications as diffuse as pregnancy contractions, headache, osteoporosis, hypoglycemia, and sedation. Most are rapidly absorbed by the vascular rich mucosa but this same vascular rich network then rapidly clears them to systemic circulation, hence their great efficacy in other systemic diseases. Little if any drug penetrates directly to the underlying sinus itself. As with oral dosing, typical sinus tissue concentration of nasally administered drugs is often in low single digit microgram/ml concentrations. Even for the limited classes of indicated drugs available, this is once again often sub-therapeutic for the sinus tissue itself.

Sinus patients will be medicated with pills and sprays for allergy and congestion. When added together it is not uncommon for a chronic patient to receive 12-24 courses of medications per year. Some may have fewer side effects, some more severe but the total number of prescriptions speaks to the continuous suffering and inadequacy associated with chronic sinusitis. Sinus patients will report general malaise, loss of work productivity and quality of life issues during episodes of disease.

After many rounds of medications, a sinus patient may be referred to a surgeon for evaluation. Current surgical options include both office and operating room procedures. In patients with severe disease Functional Endoscopic Sinus Surgery (FESS) or balloon sinuplasty are the most common surgical procedures. Both of these procedures permit access to the sinus through the nasal cavity, and theoretically increase drainage of the sinus. Neither approach is optimal, and patients often continue to suffer with the disease and its symptoms. In the clinic, balloon sinuplasty is a procedure wherein an inflatable balloon can be passed into each sinus, dilating obstructed outflow tracts. Relatively low risk and with modest recovery, this may offer some relief but rarely is curative or the final procedure. In the operating room under anesthesia, FESS can be performed wherein the sinuses can be surgically “opened” creating improved communication to the nasal cavity for drainage. Recovery can be slow over 2-4 weeks with frequent office visits required for removal of nasal packing and recurrent debridement. Cure again can be elusive, as up to 80% of patients will continue their prior pills and sprays while up to 25% will require repeat surgeries. In addition to an uncomfortable recovery and inherent surgical risks, side effects can include permanently decreased sense of taste and smell as well as Empty Sinus Syndrome.

It is desirable to find additional efficient and cost-effective methods for treating sinus disease and symptoms thereof.

### SUMMARY

A method of treating disease of a paranasal sinus, comprising piercing a wall of a sinus cavity with a needle syringe to form an opening, inserting the needle syringe containing a treatment formulation through the opening into the sinus cavity, and delivering the treatment formulation to the sinus cavity. The sinus disease can include symptoms of sinusitis as well as diagnosed disease. The paranasal sinus can be selected from the group consisting of a frontal sinus, an ethmoid sinus, a maxillary sinus and a sphenoid sinus, with a frontal sinus, a maxillary sinus or an ethmoid sinus being preferred, with an ethmoid sinus or a maxillary sinus being more preferred, and with an ethmoid sinus being particularly preferred.

A method of treating disease of a paranasal sinus, comprising piercing a wall of a sinus cavity with a needle syringe to form an opening, inserting the needle syringe into the opening, and aspirating and/or irrigating the sinus cavity.

A method of treating disease of a paranasal sinus, comprising piercing a wall of a sinus cavity with a needle syringe to form an opening, inserting the needle syringe into the opening, aspirating the sinus cavity and confirming placement of the needle in the sinus cavity.

A method of treating disease of a paranasal sinus, comprising piercing a wall of a sinus cavity with a needle syringe to form an opening, inserting the needle syringe into the opening, and irrigating the sinus cavity.

A method of treating disease of a paranasal sinus, comprising piercing a wall of a sinus cavity with a needle syringe to form an opening, inserting the needle syringe into the opening, aspirating the sinus cavity with the needle syringe to confirm placement of the needle in the sinus cavity and delivering a treatment formulation in the needle syringe to the sinus cavity.

A method of treating disease of a paranasal sinus, comprising placing a needle syringe in a native sinus opening, inserting the needle syringe containing a treatment formulation through the opening into the sinus cavity, and delivering the treatment formulation to the sinus cavity.

A method of treating disease of a paranasal sinus, comprising placing a needle syringe in a native sinus opening, inserting the needle syringe into the opening, and aspirating the sinus cavity with the needle syringe.

A method of treating disease of a paranasal sinus, comprising placing a needle syringe in a native sinus opening, inserting the needle syringe into the opening, aspirating the sinus cavity with the needle syringe and confirming placement of the needle in the sinus cavity.

A method of treating disease of a paranasal sinus, comprising placing a needle syringe in a native sinus opening, inserting the needle syringe into the opening, aspirating the sinus cavity with the needle syringe, confirming placement of the needle in the sinus cavity and delivering a treatment formulation from the needle syringe to the sinus cavity.

A method of treating disease of a paranasal sinus, comprising placing a catheter in a native sinus opening, inserting the catheter into the opening, and aspirating the sinus cavity with the catheter.

A method of treating disease of a paranasal sinus, comprising placing a catheter in a native sinus opening, inserting the catheter into the opening, aspirating the sinus cavity with the catheter and confirming placement of the catheter in the sinus cavity.

A method of treating disease of a paranasal sinus, comprising placing a catheter in a native sinus opening, inserting the catheter into the opening, aspirating the sinus cavity with the catheter, confirming placement of the catheter in the sinus cavity and delivering a treatment formulation from the catheter to the sinus cavity.

A method of treating disease of a paranasal sinus, comprising placing a needle syringe through a previously formed surgical opening, inserting the needle syringe containing a treatment formulation through the opening into a sinus cavity, and delivering the treatment formulation from the needle syringe to the sinus cavity. The sinus disease can include symptoms of sinusitis as well as diagnosed disease.

A method of treating disease of a paranasal sinus, comprising placing a needle syringe through a previously formed surgical opening, inserting the needle syringe through the opening into a sinus cavity, and aspirating the sinus cavity.

A method of treating disease of a paranasal sinus, comprising placing a needle syringe through a previously formed surgical opening, inserting the needle syringe through the opening into a sinus cavity, aspirating the sinus cavity with the needle syringe and confirming placement of the needle syringe in the sinus cavity.

A method of treating disease of a paranasal sinus, comprising placing a needle syringe through a previous formed surgical opening, inserting the needle syringe through the opening into a sinus cavity, aspirating the sinus cavity, confirming placement of the needle in the sinus cavity and delivering a treatment formulation from the needle syringe to the sinus cavity.

A method of treating disease of a paranasal sinus comprising piercing a wall of an ethmoid sinus with a needle syringe to form an opening, inserting the needle syringe into the opening, and delivering a treatment formulation into the ethmoid sinus.

A method of treating disease of a paranasal sinus comprising piercing a wall of an ethmoid sinus with a needle syringe to form an opening, inserting the needle syringe into the opening, and aspirating the ethmoid sinus.

A method of treating disease of a paranasal sinus comprising piercing a wall of an ethmoid sinus with a needle syringe to form an opening, inserting the needle syringe into the opening, aspirating the ethmoid sinus and confirming placement of the needle syringe in the ethmoid sinus.

A method of treating disease of a paranasal sinus comprising piercing a wall of an ethmoid sinus with a needle syringe to form an opening, inserting the needle syringe into the opening, aspirating the ethmoid sinus with the needle syringe, and confirming placement of the needle syringe in the ethmoid sinus and delivering a treatment formulation from the needle syringe to the ethmoid.

A method of treating disease of a paranasal sinus comprising accessing a wall of an ethmoid sinus through a native ethmoid sinus opening, piercing the wall of an ethmoid sinus with a needle syringe, inserting the needle syringe into the ethmoid sinus, aspirating the ethmoid sinus to confirm placement of the needle syringe in the ethmoid sinus, and delivering a treatment formulation from the needle syringe to the ethmoid sinus.

A method of treating disease of a paranasal sinus, comprising placing a needle syringe through a previously formed surgical opening in a ethmoid sinus, inserting the needle syringe into the opening, aspirating the ethmoid sinus, confirming placement of the needle in the ethmoid sinus and delivering a treatment formulation from the needle syringe to the ethmoid sinus.

The needle syringe can be used to aspirate fluid from the sinus and/or deliver a treatment formulation to a paranasal sinus or sinuses for the treatment of sinus disease or its symptoms. This procedure is performed at least once and can be performed in a series of procedures.

A method of delivering a drug treatment formulation to a sinus, the method comprising placing a needle syringe containing a drug treatment formulation through a nasal cavity, piercing the wall of the sinus with the needle syringe to form an opening in the sinus, inserting the needle syringe into the opening and delivering the drug treatment formulation to the sinus for the treatment of disease.

The treatment formulation may be an aqueous irrigation liquid. The treatment formulation may be a drug formulation. The drug treatment formulation may be commercially available formulation or may be a formulation specifically developed for use in a sinus cavity. The drug treatment formulation may comprise at least one drug for the treatment of the sinus cavity such as sinusitis or other conditions of the sinus cavity. The drug formulation may be an ophthalmic formulation wherein the formulation is administered directly to the sinus.

A method of delivering an ophthalmic formulation to a sinus, the method comprising placing a needle syringe containing an ophthalmic formulation into a nasal cavity, piercing a wall of the sinus with the needle syringe to form an opening in the sinus, inserting the needle syringe into the opening and delivering the ophthalmic formulation to the sinus for treatment of a sinus disease.

A method of delivering an ophthalmic formulation to a sinus, the method comprising placing a catheter in a native sinus opening, inserting the catheter into the opening, aspirating the sinus cavity with the catheter, confirming placement of the catheter in the sinus cavity and delivering the ophthalmic formulation from the catheter to the sinus for treatment of a sinus disease.

The drug formulation may be a novel formulation specifically developed and optimized for direct delivery to the sinus for treatment of disease.

This method could be used for a variety of sinus disease including acute, recurrent acute, and chronic sinusitis, allergic, infectious, non-infectious, polypoid and rhinitis.

This method can be performed on either surgically naive or post-surgical patients. This method could be performed before a patient is scheduled for surgery offsetting the time and cost of a potential surgical procedure. This method could be performed on patients in lieu of surgery, in preparation for surgery or for those still symptomatic in spite of surgery.

A method is provided for treating sinus disease by at least one direct injection of a treatment formulation into the sinus. The sinus can be accessed through the orbital wall, the oral cavity or the nasal cavity with a fine gauge needle, creating an opening in the wall, aspirating the sinus to confirm the placement of the injection in the sinus, flushing the sinus with saline and then instilling a drug formulation(s) (antibiotics, steroids, antifungal, allergy, etc.) into the sinus. Drug treatment formulations used in this procedure could be currently available prescription formulations or specifically formulated to either increase contact time with the sinus mucosa, delay clearance from the sinus cavity or increase penetration of the sinus mucosa. A single depot injection may resolve disease symptoms. If not, injections could be repeated daily or every few days until resolution of the symptoms. A method is provided for treating sinus disease by at least one direct injection of a treatment formulation into a polyp in the sinus.

The are several advantages of this procedure. Needle access injection to a sinus can be performed during an office visit and under topical anesthesia. A needle injection is a minimally invasive procedure, which does not require an operating room or sedation/anesthesia. Local injection of medication can both increase efficacy and decrease unwanted systemic side effects vs other routes of delivery. Improved treatment of disease can also reduce the need for more invasive surgical options.

This method could be adapted and used for other diseases of the respiratory system including asthma, chronic obstructive pulmonary disease, cystic fibrosis, viral and bacterial infections such as pneumonia, bronchitis, etc. For disease treatment of the lower respiratory tract the treatment formulation could be administered as a nebulized formulation, an inhaled formulation, a dry powder or a liquid formulation.

### DETAILED DESCRIPTION

Unless defined otherwise, all technical and scientific terms used herein have the same meaning as commonly understood by one of ordinary skill in the art.

The current invention describes methods of treating paranasal sinus disease and symptoms thereof. Sinusitis is a multifactorial disease characterized by inflammation of the mucous membranes lining the paranasal sinus cavities. The sinuses are air filled facial cavities which normally drain into the nasal cavity. In the disease state, they may become fluid filled if drainage is obstructed. Sinus disease can take many forms including acute, acute-recurrent, chronic sinusitis, infectious, allergic, polypoid and rhinitis. Some symptoms of sinus disease may include pain or tenderness around eyes, nose or forehead depending on the sinuses involved, headache, nasal discharge, sore throat, bad breath, nasal congestion, dental pain and fever.

The four paired paranasal sinuses are comprised of the frontal sinuses, the maxillary sinuses, the sphenoid sinuses and the ethmoid sinuses. The left and right frontal sinuses are located in the center of the forehead just above each eye. The maxillary sinuses are located behind the cheekbones near the upper jaw. The ethmoidal sinuses are located between the eyes. The sphenoidal sinuses are located behind the eyes. The sinuses are lined by pseudostratified, ciliated columnar epithelium and are generally air-filled hollow cavities. The sinuses are interconnected through small openings that drain into the nasal cavity. Drainage from paranasal sinuses converges at the osteomeatal complex for the frontal sinuses, maxillary sinuses and anterior ethmoids, while the posterior ethmoids and the sphenoid sinuses drain through the sphenoethmoidal recess. Mucus and the underlying respiratory mucous membrane protect the sinuses against infection. Mucus cleanses the delicate mucosa. It flushes out invading microorganisms and pollutants through its constant movement down the upper oropharyngeal respiratory tract where it is then swallowed and passes into the gastrointestinal tract for elimination. Mucus helps moderate the effects of humidity and temperature on the respiratory tract. There are millions of cilia that sweep back and forth on the average of 10-20 beats per second pushing the mucus along. Mucociliary clearance clears the sinuses of their secretions in less than 10 minutes. The mucus then drains from the nose to the throat in about 20-30 minutes. Ciliary function can be compromised by sinus infection, allergies, colds or other inflammatory factors hindering its clearance function.

Sinus diseases also referred to as sinonasal disease include acute, recurrent acute and chronic sinusitis, rhinitis, polypoid, infectious and allergies. Sinusitis is an inflammation of the mucous membranes lining the paranasal sinus cavities. When sinuses are healthy they are filled with air and when unhealthy can become blocked and filled with fluid resulting in painful obstruction or infection. The sinus disease can be located in any paranasal sinus including frontal sinus, the maxillary sinus, the sphenoid sinus and the ethmoid sinus.

As sinus disease is primarily a medical problem, it should primarily be treated pharmaceutically. Existing oral medications and topical nasal spray formulations are unable to adequately penetrate an intact sinus, usually delivering only microgram/ml tissue concentrations of active drug to the tissue. Patients need an alternative for targeted medical delivery which delivers sufficient tissue drug levels without systemic side effects. For many diseases, direct delivery to the organ of interest/site of pathology has provided significant advantages over systemic dosing. A small amount of medication locally delivered can provide greater efficacy with few side effects compared to systemic or peripheral delivery. This concept can be extrapolated to chronic sinus disease. It is proposed that direct intra sinus injection of drug formulations can be easily and comfortably performed, provide superior efficacy, while also minimizing or eliminating systemic side effects vs current pills or sprays.

Otolaryngologists are well trained in sinus puncture and irrigation. During routine clinic visits, it is a possible to obtain needle access of a sinus either through the roof of the mouth or preferably the nasal cavity. Saline irrigation and/or aspiration to obtain sinus culture are regularly performed and this access could also be used for direct delivery of therapeutics to the sinus cavity itself. Local targeted delivery could finally achieve therapeutic drug levels while avoiding systemic side effects.

Existing injectable antibiotics or steroids are optimized for either intravenous or intramuscular administration. The majority of these antibiotics and steroids are for systemic delivery, thus they are formulated with high amounts of active ingredient in order to deliver the appropriate amount of active for the condition being treated. A functional alternative with extensive sinonasal tissue exposure exists as prescription eye drops. Not only tested and proven safe on the delicate tissue of the eye, they also have decades of inadvertent sinonasal exposure confirming safety and tolerability. Normal eye drainage of tears (and by default, any administered eye drop) is to the nasal cavity via the nasolacrimal duct. The sinonasal tissue defined as both the nasal cavity and sinus has been exposed to every eye drop ever administered. Even though administered in small volumes, it should be noted that prior to approval and with extensive subsequent commercial use, the majority of prescription eye drops have demonstrated no sinonasal toxicity. It is proposed that administering ophthalmic formulations to the sinus provides a safe, effacious and novel method for the treatment for a range of sinus diseases. Ophthalmic formulations have been carefully developed and formulated to ensure adequate bioavailability of the active pharmaceutical ingredient (API) and to have minimal irritation to the eye. These formulations generally take the form of a solution, suspension, ointment or an emulsion, as these formulations are administered directly onto the surface of the eye. As these formulations are optimized for the stability and bioavailability, they must also be pH-balanced to avoid causing irritation to the cornea and precorneal tissue of the eye thus the pH of these formulations is close to that of tear fluid pH 7.4. There are many benefits to using ophthalmic eyedrops for sinus disease, by vastly reducing total dose and limiting exposure to a local tissue, most or all of the systemic side effects should be eliminated. For example, in the case of antibiotic treatment for sinusitis, there would be avoidance of GI upset or diarrhea and most importantly reduced or eliminated antibiotic resistance. In the case of steroidal treatment of sinusitis, reduced or eliminated GI upset would be expected, as well as reduced symptoms of dysphoria, immune suppression and osteomalacia. Similar benefits should be seen for other drug formulations including those for allergy etc.

It is reasonable to suggest that intra sinus injection will offer greater efficacy to patients suffering from sinus disease by delivering sufficient therapeutic levels of drug while at the same time reducing systemic exposure and previously unavoidable side effects. In addition to the clear advantages for patients, the ability to drastically reduce antibiotic prescriptions has the potentially to significantly impact their global overuse and rising drug resistance. As a simple injection and/or a series of low cost injections, this novel treatment may also divert significant numbers of patients from costly and modestly effective surgeries.

There are a range of diagnostic tests for sinusitis including physical examination, skin tests for allergens, CT scan of sinuses, membrane biopsy, endoscopic nasal evaluation, nasal swab test, and sinus puncture.

This method can be performed on either surgically naive or post-surgical patients. This method can be performed before a patient is scheduled for surgery offsetting the time and cost of a potential surgical procedure. This method can be performed on patients in lieu of surgery, in preparation for surgery or for those still symptomatic in spite of surgery.

Entry into a sinus can occur through a native sinus passage or opening, through a opening in a sinus wall created by a needle syringe or through a previously formed surgical opening into the sinus. Several sinus surgeries can create surgical openings into a sinus which can be used to access the sinus without the need to create a new opening. Using a small gauge needle creates a small hole in the sinus wall that will not interfere with normal sinus or nasal cavity function.

A method of treating disease of a paranasal sinus, comprising piercing a wall of a sinus cavity with a needle syringe to form an opening, inserting the needle syringe into the opening, and aspirating the sinus cavity with the needle syringe.

A method of treating disease of a paranasal sinus, comprising piercing a wall of a sinus cavity with a needle syringe to form an opening, inserting the needle syringe into the opening, and irrigating the sinus cavity.

A method of treating disease of a paranasal sinus, comprising piercing a wall of a sinus cavity with a needle syringe to form an opening, inserting the needle syringe into the opening, aspirating the sinus cavity and confirming placement in the sinus cavity.

A method of treating disease of a paranasal sinus comprising piercing a wall of a paranasal sinus with a needle syringe to form an opening, inserting the needle syringe into the opening, and delivering a treatment formulation from the needle syringe into the sinus cavity.

A method of treating disease of a paranasal sinus, comprising piercing a wall of a sinus cavity with a needle syringe to form an opening, inserting the needle syringe into the opening, aspirating the sinus cavity with a needle syringe, confirming placement in the sinus cavity and delivering the treatment formulation from the needle syringe to the sinus cavity.

A method of treating disease of a paranasal sinus, comprising accessing a sinus cavity through a native sinus opening, inserting a needle syringe containing a treatment formulation through the opening into the sinus cavity, and delivering the treatment formulation from the needle syringe to the sinus cavity.

A method of treating disease of a paranasal sinus, comprising accessing a sinus cavity through a native sinus opening inserting a needle syringe into the opening, aspirating the sinus cavity with the needle syringe.

A method of treating disease of a paranasal sinus, comprising accessing a sinus cavity through a native sinus opening inserting a needle syringe into the opening, aspirating the sinus cavity with the needle syringe and confirming placement of the needle in the sinus cavity.

A method of treating disease of a paranasal sinus, comprising accessing a sinus cavity through a native sinus opening, inserting a needle syringe into the opening, aspirating the sinus cavity with the needle syringe, confirming placement of the needle in the sinus cavity and delivering a treatment formulation from the needle syringe to the sinus cavity.

A method of treating disease of a paranasal sinus, comprising placing a catheter in a native sinus opening, inserting the catheter into the opening, and aspirating the sinus cavity with the catheter.

A method of treating disease of a paranasal sinus, comprising placing a catheter in a native sinus opening, inserting the catheter into the opening, aspirating the sinus cavity with the catheter and confirming placement of the catheter in the sinus cavity.

A method of treating disease of a paranasal sinus, comprising placing a catheter in a native sinus opening, inserting the catheter into the opening, aspirating the sinus cavity with the catheter, confirming placement of the catheter in the sinus cavity and delivering a treatment formulation from the catheter to the sinus cavity.

A method of treating disease of a paranasal sinus, comprising placing a needle syringe through a previously formed surgical opening in a sinus, inserting the needle syringe containing a treatment formulation in the needle syringe through the opening into the sinus cavity, and delivering the treatment formulation to the sinus cavity. The sinus disease can include symptoms of sinusitis as well as diagnosed disease.

A method of treating disease of a paranasal sinus, comprising placing a needle syringe through a previously formed surgical opening in a sinus, inserting the needle syringe into the opening of a sinus cavity, and aspirating the sinus cavity with the needle syringe.

A method of treating disease of a paranasal sinus, comprising piercing a wall of a sinus cavity with a needle syringe through a previous surgical opening, inserting the needle syringe into the opening of a sinus cavity, aspirating the sinus cavity to with the needle syringe and confirming placement of the needle in the sinus cavity.

A method of treating disease of a paranasal sinus, comprising piercing a wall of a sinus cavity with a needle syringe through a previously formed surgical opening, inserting the needle syringe into the opening, aspirating the sinus cavity, confirming placement of the needle in the sinus cavity and delivering a treatment formulation from the needle syringe to the sinus cavity.

The paranasal sinus can be selected from the group consisting of a frontal sinus, an ethmoid sinus, a maxillary sinus and a sphenoid sinus, with a frontal sinus, a maxillary sinus or an ethmoid sinus being preferred, with an ethmoid sinus or a maxillary sinus being more preferred, and with an ethmoid sinus being particularly preferred.

The ethmoid sinuses lie between the nasal cavity and the orbits. These sinuses are not single sacs but a collection of many small air cells. They are divided into anterior, middle and posterior air cell groups. The ethmoid bulla is the largest of the anterior ethmoid air cells. It connects to the nasal cavity via the middle meatus. Ethmoid sinusitis can be triggered by an upper respiratory infection, common cold or allergies. Entry into the ethmoid sinus can occur through a native sinus passage or opening, through an opening in a sinus wall created by a needle syringe or through a previously formed surgical opening into the ethmoid sinus. Sinus surgeries can create surgical openings into the ethmoid sinus which can then be used to access the ethmoid sinus without the need to create a new opening into the ethmoid sinus. Using a small gauge needle creates a small hole in the sinus wall that will not interfere with normal sinus or nasal cavity function.

A method of treating disease of an ethmoid sinus comprising piercing the wall of an ethmoid sinus with a needle syringe to form an opening, inserting the needle syringe into the opening, aspirating the ethmoid sinus to confirm placement of the needle syringe in the ethmoid sinus, and delivering a treatment formulation into the ethmoid sinus.

A method of treating disease of a paranasal sinus comprising piercing a wall of an ethmoid sinus with a needle syringe to form an opening, inserting the needle syringe into the opening, and aspirating the ethmoid sinus.

A method of treating disease of a paranasal sinus, comprising piercing a wall of a sinus cavity with a needle syringe to form an opening, inserting the needle syringe into the opening, and irrigating the sinus cavity.

A method of treating disease of a paranasal sinus comprising piercing a wall of an ethmoid sinus with a needle syringe to form an opening, inserting the needle syringe into the opening, aspirating the ethmoid sinus, and confirming placement of the needle syringe in the ethmoid sinus.

A method of treating disease of a paranasal sinus comprising piercing a wall of an ethmoid sinus with a needle syringe to form an opening, inserting the needle syringe into the opening, aspirating the ethmoid sinus, and confirming placement of the needle syringe in the ethmoid sinus and delivering a treatment formulation to the ethmoid.

A method of treating disease of a paranasal sinus comprising accessing a wall of an ethmoid sinus through a native sinus opening, piercing the wall of an ethmoid sinus with a needle syringe, inserting the needle syringe into the ethmoid sinus, aspirating the ethmoid sinus to confirm placement of the needle syringe in the ethmoid sinus, and delivering a treatment formulation into the ethmoid sinus.

A method of treating disease of a paranasal sinus comprising accessing a wall of an ethmoid sinus through a native sinus opening, piercing the wall of an ethmoid sinus with a needle syringe, inserting the needle syringe into the ethmoid sinus, and irrigating the ethmoid sinus.

A method of treating disease of a paranasal sinus, comprising piercing a wall of an ethmoid sinus with a needle syringe through a previous surgical opening, inserting the needle syringe into the opening, aspirating the ethmoid sinus, confirming placement of the needle in the ethmoid sinus and delivering a treatment formulation to the ethmoid sinus.

A method of treating disease of a paranasal sinus comprising accessing a wall of an ethmoid sinus with a needle syringe through a previous surgical opening, inserting the needle syringe into the opening, and irrigating the ethmoid sinus.

The needle syringe is a combination of a syringe and needle. The syringe member of the needle syringe can be either partially or completely empty for aspirating and/or irrigating a sinus cavity and the needle syringe can contain a treatment formulation for delivery to the sinus cavity for the treatment of disease. Typically, the needle on the needle syringe will range in size from 16 to 27 gauge needle.

A kit comprising multiple surgical tools. The kit includes a first surgical tool forming an opening into a paranasal sinus and contains a second surgical tool including a prefilled syringe for delivery of a treatment formulation into an opening of the paranasal sinus after the opening has been formed. A kit may include a prefilled syringe configured to attach to an endoscope for delivery of a treatment formulation to a paranasal sinus.

The treatment formulation may be a flowable medium for example: an aqueous irrigation liquid, spray or viscous gel. The treatment formulation may be a drug formulation. The drug treatment formulation may be a commercially available formulation or may be a formulation specifically developed for use in a sinus cavity. The drug treatment formulation may comprise at least one drug for the treatment of the sinus cavity such as sinusitis or other conditions of the sinus cavity. The drug treatment formulation may comprise one or more of the following: an antibiotic, a steroid, an anti-viral, an antihistamine, a monoclonal antibody, a probiotic, an anti-fungal, a mast cell stabilizer, a mucolytic, a non-steroidal anti-inflammatory drug (NSAID), a vasoconstrictor and an immunosuppressant an immune modulator, leukotriene inhibitors. Some example antibiotics include: sulfa or sulfonamide drugs, penicillin drugs macrolide drugs, aminoglycoside drugs, fluoroquinolone drugs, cephalosporin drugs and drug combinations, such as a combination of tobramycin and dexamethasone (e.g. in Tobradex®). The drug treatment formulations may contain additives such as stabilizers, surfactants, penetration enhancers, and bioadhesives. The drug formulations may contain combinations of drugs useful for the treatment of sinus disease.

Delivery of drug treatment formulations topically to the sinus would enable drug formulations to come into direct contact with the sinus and the cilia in the sinus. This localized and topical drug exposure would be an improvement over systemic drug usage. It is expected that topical application of the drug treatment formulation directly to the sinus would have an increased contact time on the surface of the cilia. Action of the drug treatment formulations on mucociliary function may be of importance to the resolution of sinus disease. Beating epithelial cilia contribute to the mucociliary clearance with ciliary beat frequency (CBF) being an important indicator of cilia function. Drug treatment formulations may also inhibit biofilm formation thus contributing to mucociliary clearance. Moderating CBF may be useful in the treatment of sinus disease as a slowing of CBF may increase contact time of drug treatment formulations and increasing of CBF may increase clearance of mucous thus aiding in disease resolution.

Treatment formulation may be developed based on attributes required for the location of the formulations. For example, a treatment formulation may contain sustained release drug formulations in a flowable medium that extends the contact time of the formulations in the sinus cavity. This formulation may slow mucociliary clearance for an intended treatment period after delivery of the treatment formulation.

For other respiratory tissues, inhibition of CBF may also yield delayed drug clearance, increased contact time and efficacy. For certain circumstances, increased CBF yielding increased clearance may also be of benefit in some diseases such as in cystic fibrosis.

A method is provided for treating sinus disease by at least one direct injection of a treatment formulation into a sinus. The sinus can be accessed through the orbital wall, the oral cavity or the nasal cavity with a fine gauge needle, creating an opening in the sinus, aspirating the sinus to confirm the placement of the needle syringe in the sinus, flushing the sinus with saline and then delivering a pharmacologic or multiple pharmacologic agents (antibiotics, steroids, allergy, etc.) from the needle syringe into the sinus. Drug treatment formulations used in this procedure could be currently available off prescription formulations or specifically formulated to increase contact time with the sinus mucosa or delayed clearance from the sinus. A single depot injection may resolve disease symptoms. If not, injections could be repeated daily or every few days until resolution of the symptoms.

A method is provided for treating sinus disease by at least one direct injection of a treatment formulation into an ethmoid sinus. The ethmoid sinus can be accessed through the orbital wall, the oral cavity or the nasal cavity with a fine gauge needle, creating an opening in the sinus, aspirating the sinus to confirm the placement of the needle syringe in the sinus, flushing the sinus with saline and then delivering a pharmacologic or multiple pharmacologic agents (antibiotics, steroids, allergy, etc.) from the needle syringe into the sinus. Drug treatment formulations used in this procedure could be currently available off prescription formulations or specifically formulated to increase contact time with the sinus mucosa or delayed clearance from the sinus. A single depot injection may resolve disease symptoms. If not, injections could be repeated daily or every few days until resolution of the symptoms.

A method is provided for treating sinus disease by at least one direct injection of a treatment formulation into an ethmoid sinus. The ethmoid sinus can be accessed through the bulla of the ethmoid with a fine gauge needle, creating an opening in the sinus, aspirating the sinus to confirm the placement of the needle syringe in the sinus, flushing the sinus with saline and then delivering a pharmacologic or multiple pharmacologic agents (antibiotics, steroids, allergy, etc.) from the needle syringe into the sinus. Drug treatment formulations used in this procedure could be currently available off prescription formulations or specifically formulated to increase contact time with the sinus mucosa or delayed clearance from the sinus. A single depot injection may resolve disease symptoms. If not, injections could be repeated daily or every few days until resolution of the symptoms.

A method is provided for treating sinus disease by at least one direct injection of a treatment formulation into a polyp in a sinus. The sinus can be accessed through the orbital wall, the oral cavity or the nasal cavity with a fine gauge needle, creating an opening in the wall, aspirating the sinus to confirm the placement of the injection in the sinus, and then instilling a drug formulation(s) (antibiotics, steroids, antifungal, allergy, etc.) into the polyp in the sinus. Drug treatment formulations used in this procedure could be currently available prescription formulations or specifically formulated to either increase contact time with the sinus mucosa, delay clearance from the sinus cavity or increase penetration of the sinus mucosa. A single depot injection may resolve disease symptoms of the polyp. If not, injections could be repeated daily or every few days until resolution of the symptoms.

As proposed, local and direct sinus injection offers many therapeutic advantages over pills and nasal sprays. The ability to directly deliver 100% of the desired dose to the sinus translates to milligram/cc tissue concentration, not microgram/cc concentration. The ability to deliver 100-1000 fold drug levels would finally enable physicians to effectively treat sinus disease compared to current dosing options. In addition to greater efficacy, direct injection of the local sinus also greatly reduces or eliminates unwanted systemic side effects. For steroids, reduction or elimination of GI upset, depression, glucose imbalance, sepsis, osteoporosis/hip fracture. For antibiotics, reduction or elimination of GI upset, diarrhea, yeast infection (female), C difficile risk, and drug resistance.

A method of delivering drug treatment formulations to a paranasal sinus comprising piercing a wall of a sinus cavity with a needle syringe to form an opening, inserting the needle syringe into the opening, aspirating the sinus cavity and confirm placement in the sinus cavity and delivering a treatment formulation to the paranasal sinus.

A method of delivering a drug treatment formulation to a paranasal sinus comprising piercing a wall of a paranasal sinus with a needle syringe to form an opening, inserting the needle syringe into the opening, irrigating the sinus cavity and delivering a treatment formulation to the paranasal sinus.

A method of delivering a drug treatment formulation to a paranasal sinus comprising piercing a wall of a paranasal sinus with a needle syringe to form an opening, inserting the needle syringe into the opening, and delivering a treatment formulation into the sinus cavity.

A method of delivering a drug treatment formulation to a paranasal sinus comprising piercing a wall of an ethmoid sinus with a needle syringe to form an opening, inserting the needle syringe into the opening, and delivering a treatment formulation into the ethmoid sinus.

A method of delivering a drug formulation to an ethmoid sinus comprising piercing an ethmoid bulla with a needle syringe forming an opening, inserting the needle syringe into the opening, and delivering a treatment formulation into the ethmoid sinus.

A method of delivering a drug formulation to an ethmoid sinus comprising piercing an ethmoid bulla with a needle syringe forming an opening, inserting the needle syringe into the opening, irrigating the ethmoid sinus and delivering a treatment formulation into the ethmoid sinus.

A method of delivering a drug formulation to an ethmoid sinus comprising piercing an ethmoid bulla with a needle syringe forming an opening, inserting the needle syringe into the opening, aspirating the ethmoid sinus and delivering a treatment formulation into the ethmoid sinus.

A method of delivering an ophthalmic formulation to a sinus, the method comprising placing a needle syringe containing an ophthalmic formulation into a nasal cavity, piercing a wall of the sinus with the needle syringe to form an opening in the sinus, inserting the needle syringe into the opening and delivering the ophthalmic formulation to the sinus for treatment of a sinus disease.

A method of treating a disease of a paranasal sinus, the method comprising, anesthetizing a bulla of a ethmoid sinus, piercing the bulla with a needle syringe to form an opening in the ethmoid sinus, injecting an irrigation solution into the sinus, aspirating the irrigation solution to confirm placement of the needle syringe, removing the syringe from the needle, attaching a syringe containing a treatment formulation and delivering the treatment formulation to the sinus. The disease can be selected from the group comprising acute, recurrent acute, chronic, infectious, allergic, polypoid and rhinitis.

Ophthalmic formulations have been carefully developed and formulated to ensure adequate bioavailability of the active pharmaceutical ingredient (API) and to have minimal irritation to the eye. It is also noted that only about 5% of the active in these formulations reach the target as up to 75% of the eyedrops are lost via nasolacrimal drainage. These formulations generally take the form of a solution, suspension, ointment or an emulsion, as these formulations are administered directly onto the surface of the eye. As these formulations are optimized for the stability and bioavailability, they must also be pH-balanced to avoid causing irritation to the cornea and precorneal tissue of the eye thus the pH of these formulations is close to that of tear fluid pH 7.4. There are many benefits to using ophthalmic eyedrops for sinus disease, by vastly reducing total dose and limiting exposure to a local tissue, most or all of the systemic side effects should be eliminated. Potential ophthalmic drops useful for injection into a sinus for the treatment of a sinus disease include allergy drugs, antibiotic drugs, nonsteroidal drugs, and corticosteroids. Potential ophthalmic drugs useful for injection into a sinus for the treatment of allergy and symptoms thereof include ketorolac tromethamine, ketotifen fumarate, loteprednol etabonate, bepotastine besilate, epinastine HCL, emedastine difumarate, alcaftadine, azelastine hydrochloride, olopatadine hydrochloride (as either 0.2%, 0.1%, 0.7%), nedocromil sodium, lodoxamide tromethamine and cromolyn sodium. Potential ophthalmic drugs useful for injection into a sinus for the treatment of sinusitis include antibiotics such as fluoroquinolones (besifloxacin, ciprofloxacin, moxifloxacin, ofloxacin, moxifloxacin, and gatifloxacin), aminoglycosides (tobramycin, gentamycin) polymyxin b combinations (polymyxinB/trimethoprim, polymyxinB/neomycin/gramicidin) and azithromycin. Ophthalmic preparations of non-steroidal anti-inflammatories (NSAIDS) may relieve some of the symptoms associated sinusitis, examples of these drugs include bromfenac, nepafenac and diclofenac. these drugs could be used in combination. Ophthalmic preparations of corticosteroids may be useful for injection into a sinus for the treatment of sinusitis such as difluprednate, prednisolone acetate, loteprednol, rimexolone, dexamethasone, fluorometholone, mometasone and hydrocortisone. Several of these drugs are used in combination such as tobramycin and dexamethasone. It is also important to note that most of these ophthalmic formulations are safe to use in children as well as adults.

As this method involves simple injection following topical anesthetic into the nasal cavity this procedure is relatively quick and painless. The common topical anesthetic drops such as proparicaine, tetracaine, benoxinate all are effective for a period of 10-30 minutes. Following administration of the topical anesthetic and allowing the anesthetic to take effect the injection is then performed within the anesthetic timeframe.

For example, if the sinus disease is a seasonal allergy, the method can be performed as the allergy season approaches (for symptomatic patients or prophylactically) and can be administered over several days or up to several weeks as necessary. In some cases, a patient will come into the ENTs office, a local topical anesthesia will be applied inside the nasal cavity, a needle syringe will be placed in the nasal cavity, and the wall of the ethmoid bulla pierced to gain access to the ethmoid sinus. The ethmoid sinus will then be aspirated and/or irrigated, and a drug treatment formulation can be delivered. This general procedure can be repeated daily as necessary until the symptoms are resolved.

If the sinus disease is chronic rhinosinusitis, the method can be performed with the onset of symptoms or if refractory to other therapies. In some cases, a patient will come into the ENTs office, a local topical anesthesia will be applied inside the nasal cavity, a needle syringe will be placed in the nasal cavity, and the wall of the ethmoid bulla pierced to gain access to the ethmoid sinus. The ethmoid sinus will then be aspirated and/or irrigated, and a drug treatment formulation can be delivered. This general procedure can be repeated daily as necessary until the symptoms are resolved.

The drug treatment formulation can be injectable, inhalable or delivery by irrigation. The treatment formulation may be a dry powder or flowable medium for example: an aqueous irrigation liquid, spray or viscous gel. The treatment formulation may be a drug formulation. The drug treatment formulation may be a commercially available formulation or may be a formulation specifically developed for use in the respiratory tract. The drug treatment formulation may comprise at least one drug for the treatment of the respiratory disease such as asthma, CF or COPD. The drug treatment formulation may comprise one or more of the following: an antibiotic, a steroid, an anti-viral, an antihistamine, a monoclonal antibody, a probiotic, an anti-fungal, a mast cell stabilizer, a mucolytic, a non-steroidal anti-inflammatory drug (NSAID), a vasoconstrictor, an immunosuppressant, an immune modulator, leukotriene inhibitors, bronchodilators, beta agonists, proteins and enzymes. Some example antibiotics include: sulfa or sulfonamide drugs, penicillin drugs. macrolide drugs, aminoglycoside drugs, fluoroquinolone drugs, cephalosporin drugs and antibiotic drug combinations, such as a combination of tobramycin and dexamethasone (e.g. in Tobradex®). The drug treatment formulations may contain additives such as stabilizers, surfactants, penetration enhancers, biologic inhibitors and bioadhesives.

Delivery of drug treatment formulations topically to the respiratory tract would enable drug formulations to come into direct contact with the tissue and the cilia. This localized and topical drug exposure would be an improvement over systemic drug usage. It is expected that topical application of the drug treatment formulation directly to the mucosa would have an increased contact time on the mucosal surface. Action of the drug treatment formulations on mucociliary function may be of importance to the resolution of a respiratory disease. Beating epithelial cilia contribute to the rapid mucociliary clearance with ciliary beat frequency (CBF) being an important indicator of cilia function. Drug treatment formulations may also inhibit biofilm formation thus contributing to mucociliary clearance. Moderating CBF may be useful in the treatment of respiratory disease as a temporary slowing of CBF may increase contact time of drug treatment formulations and increasing CBF may increase clearance of mucous, pathogens or other particles thus aiding in disease resolution.

Ophthalmic drugs currently available serve as a proof of concept for delivery to sinus tissue and for the treatment of sinus disease. Typical ophthalmic formulations contain an API generally between 0.01% to 2% of the composition, a preservative and buffering solutions. Preservatives used include benzylkonion chloride, chlorbutanol, phenylmercuric acetate and phenylmercuric nitrate. A side effect of benzykonium chloride is noted as its adverse effect on ciliary function. It is hypothesized that injecting a formulation containing benzylkonium chloride into the sinus for the treatment of sinusitis has a temporary effect of slowing CBF allowing more contact time with the active ingredient to improve symptoms of sinus infection. Drug formulations designed for delivery to the sinus for the treatment of sinus disease is possible. Drug formulations designed for delivery to the lower respiratory tract for treatment of lung disease are also contemplated. A drug formulation may take the form of a solution, a suspension, an ointment or an emulsion. The proposed formulations for delivery to the lungs or sinus may contain API in a range of about 0.001% to about 4% of the composition, a preservative such as benzylchloride in a range of about 0.01% to about 1.0% of the composition, a buffered solution close to normal physiological pH and optionally a penetration enhancer.

Example 1: Treatment of chronic sinusitis. Five (5) patients were treated for chronic sinusitis. All patients presented with chronic rhino sinusitis and were under long term care by their otolaryngologist. They were surgically naive and had failed maximal medical therapy with continued severe sinus symptoms. All patients had prior sinus CT scans available for anatomical assessment and procedure planning. All patients were treated as follows: Topical anesthetic was applied to the inside of the nasal cavity at the injection site. A syringe with 25 or 26 gauge needle was passed from the nasal cavity through the ethmoid bulla into the sinus cavity. Upon aspiration, free air was easily withdrawn demonstrating correct needle placement in the ethmoid bulla of the sinus cavity. A syringe containing 1-2 cc of saline was injected into the ethmoid sinus and direct observation of saline drainage into the nasal cavity at the osteomeatal complex was observed, demonstrating correct needle placement in the sinus cavity. A syringe containing 0.5 cc of ophthalmic Tobradex® solution (1.5 mg/cc of dexamethasone and 3 mg/cc tobramcyin) was injected into the sinus cavity. The procedure was repeated bilaterally with both left and right ethmoid sinuses injected. Bilateral injections were performed on days 1, 3, and 5 for a total of 3 treatments. The patients were followed for 1-4 weeks after final injections. All patients reported significant relief, their self-reported quality of life (QOL) symptoms were queried, and all patients reported a greater than 50% improvement above their baseline and as compared to oral medications and nasal sprays.

The foregoing description of the present invention and various aspects thereof has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

The description of a feature or features in a particular combination do not exclude the inclusion of an additional feature or features in a variation of the particular combination. Processing steps and sequencing are for illustration only, and such illustrations do not exclude inclusion of other steps or other sequencing of steps to an extent not necessarily incompatible. Additional steps may be included between any illustrated processing steps or before or after any illustrated processing step to an extent not necessarily incompatible.

The terms “comprising”, “containing”, “including” and “having”, and grammatical variations of those terms, are intended to be inclusive and nonlimiting in that the use of such terms indicates the presence of some condition or feature, but not to the exclusion of the presence also of any other condition or feature. The use of the terms “comprising”, “containing”, “including” and “having”, and grammatical variations of those terms in referring to the presence of one or more components, subcomponents or materials, also include and is intended to disclose the more specific embodiments in which the term “comprising”, “containing”, “including” or “having” (or the variation of such term) as the case may be, is replaced by any of the narrower terms “consisting essentially of” or “consisting of” or “consisting of only” (or the appropriate grammatical variation of such narrower terms). For example, a statement that something “comprises” a stated element or elements is also intended to include and disclose the more specific narrower embodiments of the thing “consisting essentially of” the stated element or elements, and the thing “consisting of” the stated element or elements. Examples of various features have been provided for purposes of illustration, and the terms “example”, “for example” and the like indicate illustrative examples that are not limiting and are not to be construed or interpreted as limiting a feature or features to any particular example. The term “at least” followed by a number (e.g., “at least one”) means that number or more than that number. The term at “at least a portion” means all or a portion that is less than all. The term “at least a part” means all or a part that is less than all.

What is claimed is:

1. A method of treating a disease of a paranasal sinus, the method comprising
piercing a wall of the paranasal sinus with a needle syringe to form an opening,
inserting the needle syringe containing a treatment formulation through the opening and into the paranasal sinus, and
delivering the treatment formulation to the paranasal sinus.

2. A method of delivering a drug treatment formulation to a sinus, the method comprising
placing a needle syringe containing a drug formulation into a nasal cavity,
piercing a wall of the sinus with the needle syringe to form an opening,
inserting the needle syringe into the opening and
delivering the drug formulation to the sinus.

3. A method of treating ethmoid sinusitis, comprising
piercing a wall of an ethmoid sinus with a needle syringe to form an opening,
inserting the needle syringe containing a treatment formulation through the opening into the ethmoid sinus and irrigating the ethmoid sinus.

4. The method of claim 1, wherein the disease is selected from the group consisting of acute, recurrent acute, chronic, infectious, allergic, polypoid sinusitis and rhinitis.

5. The method of claim 1, wherein the paranasal sinus is selected from the group consisting of a frontal sinus, an ethmoid sinus, a maxillary sinus and a sphenoid sinus.

6. The method of claim 1, wherein the treatment formulation is selected form the group consisting of an ophthalmic formulation and an irrigation solution.

7. The method of claim 1, wherein the treatment formulation is an ophthalmic formulation selected from the group consisting of an antibiotic, a steroid, an anti-viral, an antihistamine, a monoclonal antibody, a probiotic, an anti-fungal, a mast cell stabilizer, a mucolytic, a non-steroidal anti-inflammatory drug (NSAID), a vasoconstrictor, an immunosuppressant, an immune modulator, leukotriene inhibitors, bronchodilators, beta agonists, proteins, enzymes and combinations thereof.

8. The method of claim 1, wherein the treatment formulation is selected form the group consisting of an ophthalmic formulation and an irrigation solution.

9. The method of claim 2, wherein delivering the occurs multiple times until symptoms resolve.

10. The method of claim 3, wherein the disease is selected from the group consisting of acute, recurrent acute, chronic, infectious, allergic, polypoid sinusitis and rhinitis.

11. The method of claim 3, wherein the irrigating bathes the ethmoid sinus in the treatment formulation.

12. A composition for the treatment of sinusitis, said composition comprising at least one active pharmaceutical ingredient, a preservative and a buffered solution.

13. The composition of claim 12, further comprising a penetration enhancer.

14. The composition of claim 12, wherein the composition is injected into a paranasal sinus selected from the consisting of a frontal sinus, an ethmoid sinus, a maxillary sinus and a sphenoid sinus.

15. The composition of claim 12, wherein the active pharmaceutical ingredient is selected from the group consisting of an antibiotic, a steroid, an anti-viral, an antihistamine, a monoclonal antibody, a probiotic, an anti-fungal, a mast cell stabilizer, a mucolytic, a non-steroidal anti-inflammatory drug (NSAID), a vasoconstrictor, an immunosuppressant, an immune modulator, leukotriene inhibitors, bronchodilators, beta agonists, proteins, enzymes and combinations thereof.

16. The composition of claim 15, wherein the active pharmaceutical ingredients are a combination of an antibiotic and a steroid.

17. The composition of claim 12, wherein the preservatives are selected form the group consisting of benzylkonion chloride, chlorbutanol, phenylmercuric acetate and phenylmercuric nitrate.

18. A kit comprising a first surgical tool forming an opening into a paranasal sinus; and
a second surgical tool including a prefilled syringe for delivery of a treatment formulation after an opening has been formed in the paranasal sinus.

19. A kit comprising a prefilled syringe to attach to an endoscope for delivery of a treatment formulation after an opening has been formed in the paranasal sinus.
